(19) United States
(12) Patent Application Publication
(10) Pub. No.: US 2010/0317052 A1
(43) Pub. Date: Dec. 16, 2010

(54) PROCESS FOR DETECTING HELICOBACTER PYLORI USING ALIPHATIC AMIDES

(76) Inventors: Manuel António Coimbra Rodrigues Da Silva, Aveiro (PT); Sílvia Maria Da Rocha Simões Carriço, Vago (PT); Elsa Maria Carvalheiro Dias, Aveiro (PT); José Alexandre Ribeiro De Castro Ferreira, Espinho (PT)

Correspondence Address: KENEALY VAIDYA LLP, 515 EAST BRADDOCK RD SUITE B, Alexandria, VA, 22314, US

(21) Appl. No.: 12/521,568

(22) PCT Filed: Dec. 27, 2007
(86) PCT No.: PCT/IB07/55323

§ 371 Date: Jun. 27, 2009

(30) Foreign Application Priority Data
Dec. 29, 2006 (PT) 103630

(51) Int. Cl. C12Q 1/04

(52) U.S. Cl. 435/34

(57) ABSTRACT

The present invention provides a process for detecting Helicobacter pylori using aliphatic amides in which the referred amides are converted into their corresponding acids, when in contact with the bacterium. These acids are volatile or semi-volatile compounds and can be detected in the vapor phase by means of common chromatography techniques. Propionamide or butyramide can be used as amides, forming, respectively, the propionic and butyric acids. Any other non-toxic aliphatic amide can be used. The amides in use can be orally administrated as pills or as a diluted aqueous solution by both genders, any race and age, independently of the coexistence of pathologies associated or not with this bacterium. The invention is used in the development of a new non-invasive process for the detection of H. pylori in the stomach by means of analyzing exhaled air, and can be implemented in the medical and pharmaceutical industry.

### TECHNICAL FIELD OF THE INVENTION

The present invention discloses a process for detecting Helicobacter pylori using aliphatic amides.

The formation of volatile or semi-volatile compounds, namely carboxylic acids resulting from the reaction of such amides in the presence of H. pylori, confirms the presence of the bacterium in its natural environment.

Therefore, the present invention can be implemented in the medical and pharmaceutical industry.

### SUMMARY OF THE INVENTION

The present invention provides a process for detecting the presence of Helicobacter pylori using aliphatic amides. When aliphatic amides are in contact with a medium containing H. pylori, they are converted into the corresponding aliphatic acids by the action of the bacteria's aliphatic amidases. These acids are volatile or semi-volatile compounds and can be identified and quantified in the vapor phase which is associated with the medium where the bacterium is present.

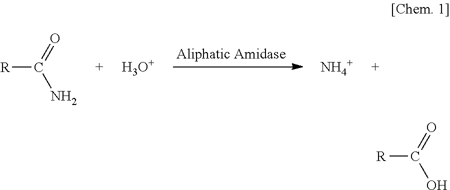

Since other bacteria with amidase activity are seldom in the stomach flora, the detection of specific aliphatic acids, normally absent in certain rates of exhaled air, is an indicator of gastroduodenal infection by Helicobacter pylori. Therefore, the present invention sets the basis for the development of a non-invasive detection process of H. pylori in the stomach, even in asymptomatic situations.

### BACKGROUND OF THE INVENTION

H. pylori is a Gram-negative bacterium which colonizes the stomach mucous of about two thirds of the world population and 75% of the Portuguese population. This bacterium is associated to the development of gastric pathologies, being considered as the main cause of type B chronic gastritis which leads to peptic ulcer and gastric carcinoma. Firstly, the H. pylori damages the mucous layer that covers the stomach epithelium. After damaging this layer, it reaches the mucus' productive cells within the stomach epithelium, damaging them and causing an inflammatory reaction, which might lead in certain cases to gastritis and subsequently to a gastric ulcer. The symptoms are felt when the infection develops into gastric ulcer.

The tests presently available to detect H. pylori might be invasive or non-invasive, from the patient's point of view. The invasive tests, such as the endoscopy, are uncomfortable and painful. The quantitative and/or qualitative determination of the anti-H. pylori IgG antibodies in biological fluids such as serum, gastric juice, saliva and urine (Versalovic, J. ‘Helicobacter pylori: Pathology and Diagnostic Strategies’, Am. J. Clin. Pathol. 2003, 119, 403-412) is also uncomfortable and expensive, although less painful for the patient than an endoscopy.

Alternatively, some non-invasive tests such as the ¹³C-urea breath test have been carried out. In order to develop this test, the patient swallows a ¹³C-urea dose. If H. pylori is present in the stomach, the urease will hydrolyze the urea, being labeled carbon dioxide (¹³CO₂) and ammonia released. ¹³CO₂ enters the blood circulation and flows into the lungs, being released through exhaled air. The determination is made by means of measuring the ¹³CO₂/¹²CO₂ ratio in exhaled air samples collected, providing this ratio a quantitative indicator of the Helicobacter pylori infection (European Medicines Agency, http://www.emea.eu.int). However this test is costly, since ¹³C-urea is relatively expensive.

Besides the urease, it has been recently disclosed that H. pylori also comprises aliphatic amidases which allow its colonization in the stomach (Bury-Moné, S. et al., ‘Presence of Active Aliphatic Amidases in Helicobacter Species Able To Colonize the Stomach’, Infect. Immun., 2003, 71, 5613-5622). This observation is the basis of the present invention's motivation, taken that the aliphatic amidases activity on the provided short-chain aliphatic amides produces ammonia and acids thereof. For instance, the administration of propionamide (R═CH₂—CH₃) or butyramide (R═CH₂—CH₂—CH₃) originates the formation of propionic acid and butyric acid, respectively. The increase of the quantity of these acids in the vapor phase reveals the presence of the bacterium.

The present invention, which configures a basis for the development of a non-invasive detection process such as the breath test of the urease, has the advantage of providing the use of an active ingredient 100 times less expensive if propionamide is chosen or, if butyramide is chosen, the use of an active ingredient 400 times less expensive.

The present invention, as well as the anti-H. pylori IgG antibodies' identification and the urease breath test, can be used as a screening test for H. pylori infection. Due to its simplicity in operational terms, it can be used in a large number of people, including asymptomatic patients for epidemiological studies, in an attempt to eradicate H. pylori from target populations as a means to prevent gastric disorders.

Therefore, the invention has the objective to provide a fast, efficient, and cost-effective means for the non-invasive detection of Helicobacter pylori in individuals.

### GENERAL DESCRIPTION OF THE INVENTION

The present invention provides a process for detecting the presence of Helicobacter pylori by means of using aliphatic amides.

The aliphatic amides, in contact with the H. pylori medium, are converted into the corresponding aliphatic acids by action of the aliphatic amidases of the bacterium. These acids are volatile or semi-volatile compounds that can be identified and quantified in the vapor phase associated to the medium where the bacterium is present.

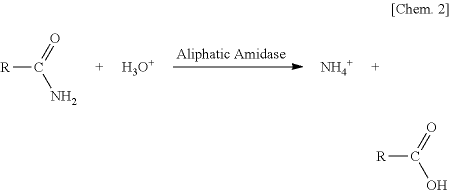

Since other bacteria with amidase activity are seldom in the stomach flora, the detection of specific aliphatic acids, normally absent in certain rates of exhaled air, is an indicator of gastroduodenal infection by Helicobacter pylori.

Therefore, a source of short-chain aliphatic amides is provided in a first phase, being the amides used to screen the presence of H. pylori in individuals by means of oral administration of formulations containing such molecules.

These formulations are made available as pills, tablets, capsules, lozenges, or as a diluted aqueous solution.

In a second phase, a research on exhaled gases of the carboxylic acids corresponding to the added amides is carried out. These acids are derived from the aliphatic amidases' activity of H. pylori on the amides administrated in the previous step of the process.

In vitro ex vivo experimental tests were carried out in H. pylori cultures from collections and clinical isolates. The results obtained indicate that after adding propionamide and/or butyramide to the medium containing the H. pylori, the formation of the respective acids takes place, occurring in the vapor phase in quantities that allow their detection and quantification through current gas chromatography techniques coupled to mass spectrometry.

Two aspects considered crucial for the future implementation of the present methodology were also confirmed: i) the amides are not hydrolyzed at the stomach's acidity conditions, therefore, not forming spontaneously the acids under these conditions, and ii) there is no formation of carboxylic acids in the medium containing the H. pylori if the amides are not added. Therefore, it can be concluded that only when H. pylori and an aliphatic amide are simultaneously present occurs the formation of the corresponding acid by the action of the bacterium aliphatic amidase.

### DETAILED DESCRIPTION OF THE INVENTION

### 1. Characterization and Administration of the Amides Used

The experimental tests were performed in vitro ex vivo in H. pylori cultures from collection and clinical isolates enriched in liquid medium (F12) under microaerophilic conditions.

The amides used in the scope of the present invention are characterized in that they are short-chain aliphatic amides. The amides used herein were propionamide and butyramide, purchased from Sigma-Aldrich, under reference numbers 143936 and 19240, respectively. According to the supplier, the purity level of propionamide was of 97% and butyramide was of 98%.

These compounds were administrated as diluted aqueous solutions (0.5 mL) to the liquid culture media (25 mL), 120 mL glass flasks hermetically sealed and encapsulated with a cap containing a septum.

### 2. Detection of the Formation of the Carboxylic Acids Produced

2.1-Collection of the Carboxylic Acids Present in the Vapor Phase Surrounding the Bacterium

The collection of the acids that were formed after the amide administration described in 1 was carried out after acidification of the medium with acid tartaric (mimetizing the stomach's pH) and exposing to the flask's vapor phase for 5 minutes at 37° C. a solid-phase microextraction fiber (SPME) coated with carbowax/divinilbenzene (65 μm thickness).

2.2-Search for the Carboxylic Acids Present in the Vapor Phase that Surrounds the Bacterium

The search in vapor phase for the carboxylic acids generated by the bacterium's aliphatic amidases activity on the amides administrated was carried out by means of gas chromatography using a capillary column of intermediate polarity.

The detection was performed by mass spectrometry comprising 3 scan cycles with selective ion monitoring (SIM) the m/z=74 for the propionic acid and m/z=88 for butyric acid.

### EXAMPLES

The administration of 0.5 mL of a 20 mM propionamide solution in a flask comprising the medium enriched with H. pylori, after 60 minutes incubation at 37° C., gave a chromatographic area of 7.0×10⁵ corresponding to the propanoic acid.

The administration of 0.5 mL of a 20 mM propionamide solution in a flask comprising only the medium, without any bacteria addition, following 60 minutes incubation at 37° C., did not produce any chromatographic peak corresponding to propanoic acid.

The administration of 0.5 mL of a 20 mM butiramide solution in a flask comprising the medium enriched with H. pylori, after 60 minutes incubation at 37° C., gave a chromatographic area of 6.5×10⁵ corresponding to the butyric acid.

The administration of 0.5 mL of a 20 mM butiramide solution in a flask comprising only the medium, without any bacteria addition, following 60 minutes incubation at 37° C., did not derive any chromatographic peak corresponding to butyric acid.

1. A process for detecting Helicobacter pylori in individuals, comprising the administration of amides and detection of the corresponding volatile or semi-volatile carboxylic acids in the exhaled gases of those individuals, resulting from the reaction of amidases produced by H. pylori.

2. The process for detecting Helicobacter pylori in individuals according claim 1 comprising:
a) administrating an amide to the individual;
b) measuring the corresponding carboxylic acid, resulting from the H. pylori reaction on the amide administrated during the administrating.

3. The process for detecting Helicobacter pylori in individuals according to the claim 2, wherein the amides are short-chain aliphatic amides.

4. The process for detecting Helicobacter pylori in individuals according to claim 3 wherein the amides are pro-pionamide or butiramide.

5. The process for detecting Helicobacter pylori in individuals according to claim 4, wherein the amides are administrated as pills, tablets, capsules, lozenges, or as a diluted aqueous solution.

6. The process for detecting Helicobacter pylori in individuals according to claim 2, further comprising scanning for volatile or semi-volatile carboxylic acids including a maximum of three steps:
a) Collecting exhaled air;
b) Separating the volatile or semi-volatile components by gas chromatography;
c) Detecting the carboxylic acids by mass spectrometry.

7. Use of the process for detecting Helicobacter pylori in individuals according to claim 6, wherein the use aims for screening of the bacterium's presence in individuals.

8. Use of the process for detecting Helicobacter pylori in individuals according to claim 5 wherein the use aims for screening of the bacterium's presence in individuals.

9. Use of the process for detecting Helicobacter pylori in individuals according to claim 4 wherein the use aims for screening of the bacterium's presence in individuals.

10. Use of the process for detecting Helicobacter pylori in individuals according to claim 3 wherein the use aims for screening of the bacterium's presence in individuals.

11. Use of the process for detecting Helicobacter pylori in individuals according to claim 2 wherein the use aims for screening of the bacterium's presence in individuals.

12. Use of the process for detecting Helicobacter pylori in individuals according to claim 1 wherein the use aims for screening of the bacterium's presence in individuals.
